(19) United States
(12) Patent Application Publication
(10) Pub. No.: US 2005/0177048 A1
(43) Pub. Date: Aug. 11, 2005

(54) Wireless heartbeat signal receiving module with anti-electromagnetic shielding

(76) Inventor: Yu-Yu Chen, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/775,108

(22) Filed: Feb. 11, 2004

(51) Int. Cl. A61B 5/0402

(52) U.S. Cl. 600/509

(57) ABSTRACT

A wireless heartbeat signal receiving module includes a shielding box made of a metal material without magnetic permeability and covering a receiver antenna on a circuit base inside the module. The shielding box is electrically connected to a ground path on the circuit base, so as to effectively isolate the receiver antenna and a heartbeat signal received via the receiver antenna from external interference signals. The shielding box is provided on a sidewall with a through hole oriented to a corresponding wireless heartbeat detecting and emitting unit that emits the heartbeat signal. The shielding box may be alternatively expanded to also cover an RF signal receiving circuit on the circuit base or to cover all areas on the circuit base.

### FIELD OF THE INVENTION

The present invention relates to an interference-proof wireless heartbeat signal receiving module, and more particularly to a wireless heartbeat signal receiving module that has anti-electromagnetic shielding to effectively isolate the module from external interference signals.

### BACKGROUND OF THE INVENTION

There are various types of body building devices and exercisers developed for people who live in the busy modern commercial society and require appropriate exercises. For a person to accurately control his actual amount of exercise and personal physical condition, various types of body signal sensing devices have been researched and developed.

For example, there is a heartbeat-detecting device frequently used to detect a user's heartbeat pulse signal. The currently commercially available heartbeat-detecting devices may be further divided into two types, namely, wired and wireless heartbeat-detecting devices. Due to its convenience for use without being restricted by any signal transmission line, the wireless heartbeat-detecting device has been widely used in different places.

Among various wireless heartbeat-detecting devices used with bodybuilding apparatuses, there is a chest type wireless heartbeat detecting and emitting unit. This unit is designed for fitting on a user's chest and mainly includes two sensing electrodes, a heartbeat detector, and an emitting unit. The heartbeat signal emitted from the chest type wireless heartbeat detecting and emitting unit is received by a receiving module that is located within an effective signal-receiving range of the emitting unit. The receiving module typically includes a display unit for indicating the detected heartbeat signal.

However, in the practical use of the chest type wireless heartbeat detecting and emitting unit with an exerciser having a wireless heartbeat receiving module provided therewith, it is found the receiving module is easily subject to interferences by various noises frequently come from the following interference sources:

(a) The display unit included in the receiving module scans and lights the pulse to result in signal interference.

(b) Brushes of motors provided in the bodybuilding apparatus and neighboring locations tend to produce sparks and magnetic fields during operation of the motors to result in signal interference.

(c) Various kinds of electronic circuit switches (including semiconductor elements, such as SCR, MOS-FET, etc.) in the bodybuilding apparatuses tend to produce noises to result in signal interference.

All the above-mentioned interference sources will adversely influence the performance of the wireless heartbeat receiving module in wirelessly receiving a heartbeat signal, resulting in different degrees of errors in the received heartbeats, failure in detecting the heartbeat, shortened effective signal receiving range, or even failure in receiving any heartbeat signal.

In conventional signal interference shielding techniques, magnetically permeable metal materials are widely used as the shielding material. For example, switching power supply, RF amplifier, and video signal processing circuit all use a magnetically permeable metal housing to shield from signal interference. This is because the magnetically permeable metal material absorbs all noises to avoid external interference. However, the shielding made of the magnetically permeable metal also causes attenuation of the received signal.

Therefore, it is necessary to develop a signal receiving module that has good signal interference shielding to solve the above-mentioned problem about the signal interference.

### SUMMARY OF THE INVENTION

A primary object of the present invention is to provide a wireless heartbeat signal receiving module with anti-electromagnetic shield. To achieve this object, the wireless heartbeat signal receiving module of the present invention includes a circuit base, on which a receiver antenna is provided and covered by a shielding box, so that the receiver antenna is isolated from external interference. Alternatively, the shielding box may be expanded to also cover an RF signal receiving circuit on the circuit base, or to cover all areas on the circuit base.

Another object of the present invention is to provide a wireless heartbeat signal receiving module that provides improved orientation. To achieve this object, the wireless heartbeat signal receiving module of the present invention includes a shielding box for covering a receiver antenna of the module, and the shielding box is provided on one sidewall oriented to a corresponding chest type wireless heartbeat detecting and emitting unit with a through hole. A heartbeat signal emitted from the wireless heartbeat detecting and emitting unit passes through the through hole and is then received by the receiver antenna. In this manner, the receiver antenna has an improved orientation to the wireless heartbeat detecting and emitting unit.

A further object of the present invention is to provide a wireless heartbeat signal receiving module that has simple structural design adapted to prevent signal interference by neighboring users, so that a plurality of the modules mounted on closely arranged bodybuilding apparatuses may work without the problem of signal interference.

To achieve the above objects, in accordance with the present invention, there is provided wireless heartbeat signal receiving module with anti-electromagnetic shield. The wireless heartbeat signal receiving module includes a shielding box made of a metal material without magnetic permeability and covering a receiver antenna on a circuit base inside the module. The shielding box is electrically connected to a ground path on the circuit base, so as to effectively isolate the receiver antenna and a heartbeat signal received via the receiver antenna from external interference signals. The shielding box is provided on a sidewall with a through hole oriented to a corresponding wireless heartbeat detecting and emitting unit that emits the heartbeat signal. The shielding box may be alternatively expanded to also cover an RF signal receiving circuit on the circuit base or to cover all areas on the circuit base.

Preferably, the shielding box is provided on one sidewall with a through hole, and the heartbeat signal emitted from said wireless heartbeat detecting and emitting unit passes through the through hole before being received by said receiver antenna. The through hole improves the orientation of the receiver antenna to the wireless heartbeat detecting and emitting unit.

In a preferred embodiment of the present invention, the wireless heartbeat signal receiving module is internally provided with an adjustable powdered iron core antenna or an adjustable capacitor. In the event an inductance or a capacitance of the receiver antenna is changed due to a shielding box provided in the present invention, the inductance or the capacitance could be corrected to obtain a correct resonance frequency.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a perspective view showing the relation between a wireless heartbeat signal receiving module with anti-electromagnetic shield according to a first embodiment of the present invention and a corresponding wireless heartbeat detecting and emitting unit;

FIG. 2 is an enlarged perspective view of the wireless heartbeat signal receiving module of FIG. 1 showing an internal structure thereof;

FIG. 3 is a partially exploded perspective view of FIG. 2;

FIG. 4 is a block diagram of a control circuit for the present invention;

FIG. 5 is a wave pattern of a signal resonance center frequency fc1 of a receiver antenna of the present invention;

FIG. 6 is an exploded perspective view of a second embodiment of the present invention, in which a shielding box defines a space to cover not only the receiver antenna, but also an RF signal receiving circuit on the circuit base; and

FIG. 7 is an exploded perspective view of a third embodiment of the present invention, in which a shielding box defines a space to cover not only the receiver antenna and the RF signal receiving circuit, but also all other areas on the circuit base.

### DETAILED DESCRIPTION OF THE PREFERRED EMBODIMENTS

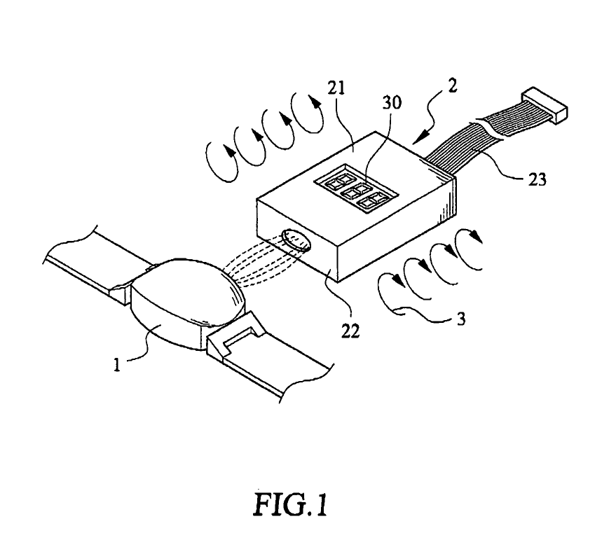

Please refer to FIG. 1 that is a perspective view showing the relation between a wireless heartbeat signal receiving module with anti-electromagnetic shield 2 according to a first embodiment of the present invention and a corresponding wireless heartbeat detecting and emitting unit 1. As shown, the wireless heartbeat detecting and emitting unit 1 is of a chest type for wearing on a user's chest to detect the user's heartbeat signal and wirelessly emitting the detected heartbeat signal to the wireless heartbeat signal receiving module 2 that is located within an effective range of receiving the emitted signal.

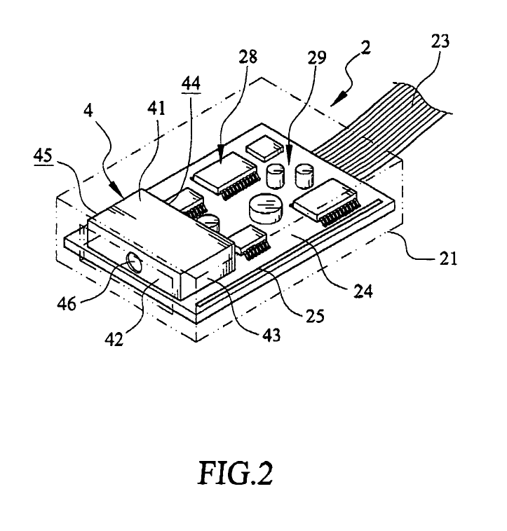

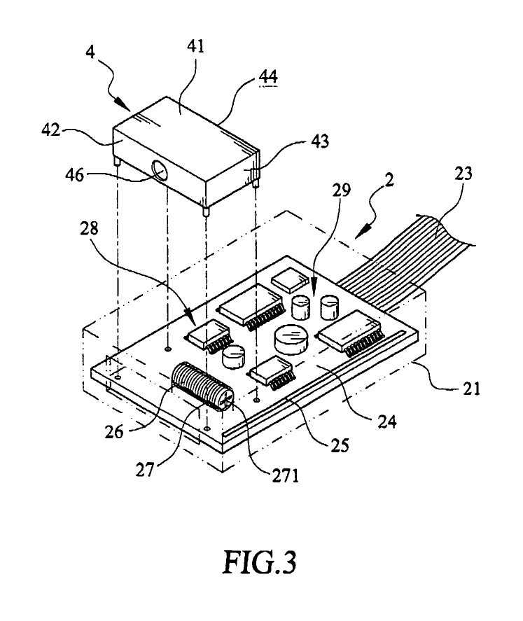

Please also refer to FIG. 2 that is an enlarged perspective view of the wireless heartbeat signal receiving module 2 of FIG. 1, and to FIG. 3 that is a partially exploded perspective view of FIG. 2. As shown, the wireless heartbeat signal receiving module 2 includes a case 21, a front end 22 of which is oriented to the wireless heartbeat detecting and emitting unit 1 to enable an enhanced signal receiving effect. With the present invention, the wireless heartbeat signal receiving module 2 is able to receive a user's heartbeat signal via a receiver antenna 26 while the latter is isolated from external interference signals.

In a space defined by the case 21 of the wireless heartbeat signal receiving module 2, there is provided a circuit base 24. At least one ground path 25 is provided on the circuit base 24.

A receiver antenna 26 is provided in the case 21 of the wireless heartbeat signal receiving module 2 close to the front end 22 thereof. The receiver antenna 26 may be a copper coil wound around a powdered iron core 27.

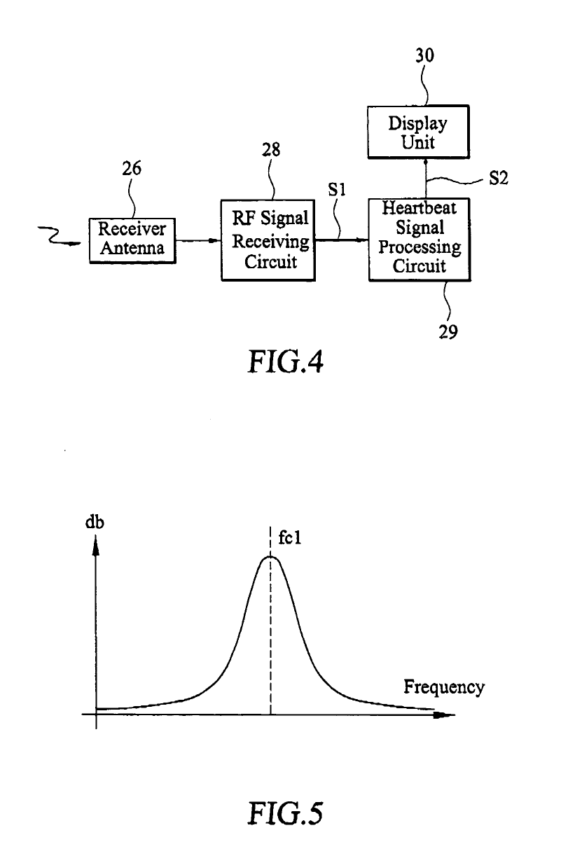

On the circuit base 24, there are provided a control circuit and other related electronic components, such as resistors, capacitors, integrated circuit (IC) components, etc. FIG. 4 is a block diagram of the control circuit.

The control circuit may include an RF signal receiving circuit 28, which receives via the receiver antenna 26 a heartbeat signal emitted from the wireless heartbeat detecting and emitting unit 1, a heartbeat signal processing circuit 29 for processing a heartbeat signal S1 received by the RF signal receiving circuit to produce a heartbeat pulse signal S2, and a display unit 30 for receiving the produced heartbeat pulse signal S2 to indicate a user's heartbeat condition. The heartbeat pulse signal S2 may be further sent to another control unit (not shown) via signal wires 23 for the purpose of data recording, statistics, etc.

In a preferred embodiment of the present invention, the wireless heartbeat signal receiving module 2 may be internally provided with an adjustable powdered iron core antenna or an adjustable capacitor. In the event an inductance or a capacitance of the receiver antenna is changed due to a shielding box provided in the present invention, the inductance or the capacitance could be corrected to obtain a correct resonance frequency.

For example, in the embodiment illustrated in FIG. 3, the receiver antenna 26 is wound around a powdered iron core 27, which includes an adjustable end 271 for adjusting a signal resonance center frequency fc1 of the receiver antenna 26, so that the signal resonance center frequency fc1 is the same as an emission frequency of the wireless heartbeat detecting and emitting unit 1 (see FIG. 5) to increase a signal to noise ratio (S/N) of the received heartbeat signal. It is also possible to adjust the signal resonance center frequency fc1 of the receiver antenna 26 by adjusting the capacitance in the RF signal receiving circuit 28.

Alternatively, the powdered iron core 27 wound by the receiver antenna 26 may be provided without the adjustable end 271 and in the form of a fixed antenna. That is, the fixed receiver antenna 26 is pre-adjusted to a correct signal resonance center frequency fc1 before the wireless heartbeat signal receiving module 2 is finished and delivered from the manufacturer's plant.

A shielding box 4 is fixed to a top of the circuit base 24 and defines a space to cover the receiver antenna 26. The shielding box 4 is electrically connected to the ground path 25 on the circuit base 24, such that the receiver antenna 26 and the heartbeat signal received via the receiver antenna 26 are isolated from external interference signals.

The shielding box 4 may be made of a metal material without magnetic permeability, such as copper, aluminum, zinc, etc. The shielding box 4 includes a top 41, and four sidewalls 42, 43, 44, and 45 perpendicularly downward extended from four sides of the top 41. In the first embodiment shown in FIGS. 2 and 3, the top 41 and the four sidewalls 42, 43, 44, and 45 of the shielding box 4 together defines a space large enough for covering the receiver antenna 26 therein.

The sidewall 42 of the shielding box 4 is oriented to the wireless heartbeat detecting and emitting unit 1 and provided at a predetermined position with a through hole 46. The heartbeat signal emitted from the wireless heartbeat detecting and emitting unit 1 passes through the through hole 46 before being received by the receiver antenna 26. The provision of the through hole 46 on the shielding box 4 improves the orientation of the receiver antenna 26 to the wireless heartbeat detecting and emitting unit 1.

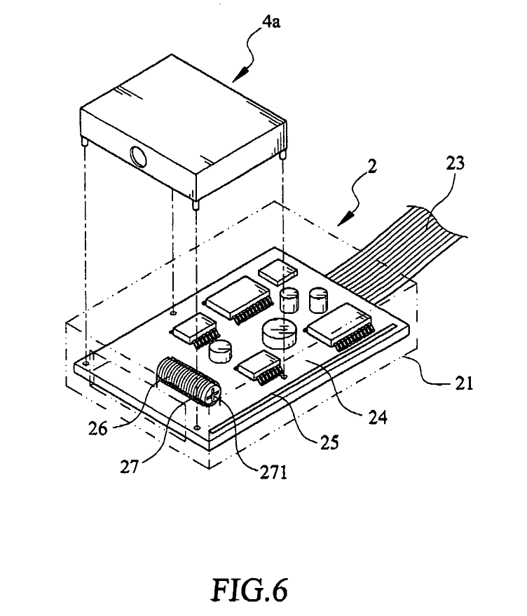

FIG. 6 is an exploded perspective view of a second embodiment of the present invention. In the second embodiment, there is provided a shielding box 4a that defines a space to cover not only the receiver antenna 26, but also the RF signal receiving circuit 28 on the circuit base 24. Therefore, the RF signal receiving circuit 28 is also completely protected against external interference signals.

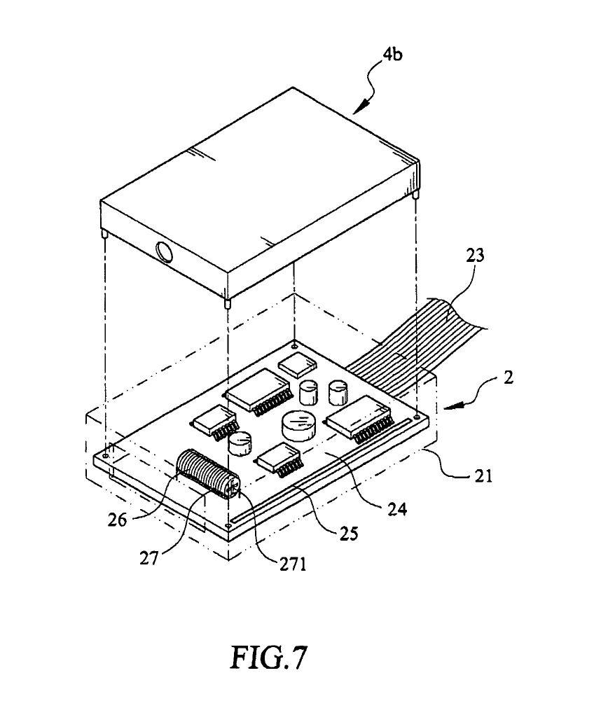

FIG. 7 is an exploded perspective view of a third embodiment of the present invention. In the third embodiment, there is provided a shielding box 4b that defines a space to cover not only the receiver antenna 26 and the RF signal receiving circuit 28, but also all other areas on the circuit base 24. The shielding box 4b not only provides complete protection against external interference signals, but also makes the production of the wireless heartbeat signal receiving module 2 easier.

The present invention has been described with some preferred embodiments thereof and it is understood that many changes and modifications in the described embodiments can be carried out without departing from the scope and the spirit of the invention that is to be limited only by the appended claims.

1. A wireless heartbeat signal receiving module with anti-electromagnetic shielding, said module being located in the vicinity of a wireless heartbeat detecting and emitting unit for receiving a user's heartbeat signal detected by and wirelessly emitted from said wireless heartbeat detecting and emitting unit, said module comprising:
a circuit base located inside a case of said module and being provided thereon with at least one ground path;
a receiver antenna for detecting said heartbeat signal emitted from said wireless heartbeat detecting and emitting unit;
an RF signal receiving circuit for receiving via said receiver antenna said heartbeat signal emitted from said wireless heartbeat detecting and emitting unit;
a heartbeat signal processing circuit for processing said heartbeat signal received by said RF signal receiving circuit to produce a heartbeat pulse signal; and
a shielding box fixed to a top of said circuit base and defining a space for covering said receiver antenna, said shielding box being electrically connected to said ground path on said circuit base, such that said receiver antenna and said heartbeat signal received via said receiver antenna are isolated from external interference signals.

2. The wireless heartbeat signal receiving module with anti-electromagnetic shielding as claimed in claim 1, wherein said shielding box is provided on one sidewall with a through hole, and said heartbeat signal emitted from said wireless heartbeat detecting and emitting unit passes through said through hole before being received by said receiver antenna.

3. The wireless heartbeat signal receiving module with anti-electromagnetic shielding as claimed in claim 1, wherein said receiver antenna includes a powdered iron core and a copper coil wound around said powdered iron core, said powdered iron core including an adjustable end for adjusting a signal resonance center frequency of said receiver antenna to be the same as an emitting frequency of said wireless heartbeat detecting and emitting unit.

4. The wireless heartbeat signal receiving module with anti-electromagnetic shielding as claimed in claim 1, wherein said shielding box includes a top and four sidewalls perpendicularly downward extended from four sides of said top, and said top and said four sidewalls together define said space of said shielding box for covering said receiver antenna.

5. The wireless heartbeat signal receiving module with anti-electromagnetic shielding as claimed in claim 1, wherein said space defined by said shielding box also covers said RF signal receiving circuit on said circuit base.

6. The wireless heartbeat signal receiving module with anti-electromagnetic shielding as claimed in claim 1, wherein said space defined by said shielding box covers all areas on said circuit base.

7. The wireless heartbeat signal receiving module with anti-electromagnetic shielding as claimed in claim 1, wherein said shielding box is made of a metal material without magnetic permeability selected from the group consisting of copper, aluminum, and zinc.
